(19) United States
(12) Patent Application Publication
(10) Pub. No.: US 2003/0158168 A1
(43) Pub. Date: Aug. 21, 2003

(54) Composition for combined use of aromatase inhibitors

(76) Inventors: Giorgio Massimini, Milan (IT); Dinesh Purandare, Branchburg, NJ (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/343,595

(22) PCT Filed: Jul. 27, 2001
(86) PCT No.: PCT/EP01/08707

(51) Int. Cl. A61K 31/56

(52) U.S. Cl. 514/182; 514/178

(57) ABSTRACT

A method of treating a human being suffering from an hormone-dependent disorder comprising administering to said human being a steroidal aromatase inhibitor e.g. exemestane of formestane and a non-steroidal aromatase inhibitor e.g. fadrozole or vorozole, in amounts effective to produce a superadditive therapeutic effect.

The present invention concerns the treatment of hormone dependent disorders, in particular estrogen dependent disorders.

Endocrine-deprivation therapy of hormone dependent disorders is based on the knowledge that certain tumors require estrogen for their growth. An attractive strategy of hormone deprivation is, therefore, to use drugs which block estrogen biosynthesis. In this connection, the use of aromatase inhibitors, which are able to inhibit the reaction which transforms androgens into estrogens by creating an aromatase ring in the steroid molecule, is now well established. The novel aromatase inhibitors are better tolerated than first-generation aromatase inhibitors. However, administration to a patient of therapeutically effective amounts of aromatase inhibitors can cause side effects. The major toxicities are for instance lethargy, hot flashes, rash, transient leukopenia, dizzines, nausea, constipation and vomiting.

It is worth noting that the influence of those side effects, although “mild”, on the quality of life may be an important factor in determining the acceptance of an aromatase inhibitor e.g. in adjuvant therapy, where such therapy is prolonged.

Moreover, whilst substantial numbers of patients with hormone dependent disorders derive benefit from aromatase inhibitors, remission in responding patients, as with other forms of endocrine therapies, is not generally permanent and disease may occur. Since aromatase inhibitors achieve their major benefit by causing estrogen deprivation, many of the mechanism by which resistance occur are likely to be shared by other forms of endocrine deprivation. These, for instance, may include the outgrowth of hormone insensitive cells and ineffective estrogen suppression and/or endocrine compensation. Since aromatase inhibitors in hormonal therapy of cancers, e.g. breast, are intended for long lasting use, toxicity, even if mild and reversible, and resistance are problematic.

The present invention provides a method for treating hormone, in particular sex steroid and specifically estrogen, dependent disorders, which overcomes the above problems.

The inventors of the present invention have found that a combination therapy of hormone dependent disorder, comprising a therapeutically effective amount of a steroidal aromatase inhibitor and a therapeutically effective amount of a non-steroidal aromatase inhibitor, can produce a therapeutic effect which is greater than that obtainable by single administration of a therapeutically effective amount of either a sole steroidal aromatase inhibitor or a sole non-steroidal aromatase inhibitor.

Similarly they have found that a combination therapy of an hormone dependent disorder, comprising a therapeutically sub-effective amount of a steroidal aromatase inhibitor and a therapeutically sub-effective amount of a non-steroidal aromatase inhibitor, can produce substantially the same therapeutic effect, which is obtainable by single administration of a therapeutically effective amount of either a steroidal aromatase inhibitor or a non-steroidal aromatase inhibitor. The most important, they have found that such newly obtained therapeutic effect is not paralleled by the toxic effects and resistance phenomena, otherwise caused by single administrations of either therapeutically effective amounts of a steroidal aromatase inhibitor or therapeutically effective amounts of a non-steroidal aromatase inhibitor.

In view of the above, the effectiveness of the hormonal therapy is significantly increased without a parallel increased toxicity and occurrence of resistance. In other words, the combined therapy of the present invention enhances the therapeutic effects of the aromatase inhibitors and thus yields more effective and less toxic treatment for hormone-dependent disorders.

Accordingly, the present invention provides a new and valuable tool in the therapy of sex steroid hormone, in particular estrogen, dependent disorders. The advantages provided by the present invention can be appreciated by their preferred features, described herebelow.

Examples of such disorders are cancers, e.g. breast, cervical, ovarian and endometrial cancers. However such disorder is preferably breast cancer in a human being, in particular a female.

Accordingly, the present invention provides, as a first object, a pharmaceutical composition comprising a steroidal aromatase inhibitor and a non-steroidal aromatase inhibitor, having a synergistic or superadditive therapeutic activity against a sex steroid hormone-dependent disorder.

The present invention also provides the use of a steroidal aromatase inhibitor in the manufacture of a pharmaceutical composition for treatment of a sex steroid hormone-dependent disorder, the treatment additionally comprising the administration of a composition comprising a non-steroidal aromatase inhibitor, in amounts effective to produce a superadditive effect.

Examples of steroidal aromatase inhibitors according to the invention are exemestane, formestane and atamestane.

Examples of non-steroidal aromatase inhibitors according to the invention are fadrozole, letrozole, vorozole and anastrozole.

Preferred examples of steroidal aromatase inhibitors according to the invention are exemestane and formestane, in particular exemestane.

Preferred examples of non-steroidal aromatase inhibitors according to the invention are fadrozole, anastrozole and letrozole, in particular anastrozole and letrozole.

The term “steroidal aromatase inhibitor” is meant to comprise both a single steroidal aromatase inhibitor or a mixture of two or three, preferably two, steroidal aromatase inhibitors as defined above. Preferably the single steroidal aromatase inhibitor, or one of the component of the mixture, is exemestane.

The term “non-steroidal aromatase inhibitor” is meant to comprise both a single non-steroidal aromatase inhibitor or a mixture of two or three, preferably two, non-steroidal aromatase inhibitors as defined above. Preferably a single non-steroidal aromatase inhibitor is used, in particular anastrozole or letrozole.

The aromatase inhibitors cited herein are well known products, which are cited for instance in Cancer-Treat-Res.: 94, 231-254, 1998 and WO 99/30708.

The present invention also provides a product comprising a steroidal aromatase inhibitor and a non-steroidal aromatase inhibitor, as combined preparation for simultaneous, separate or sequential administration, in amounts to produce a synergistic or superadditive therapeutic activity against a sex steroid hormone-dependent disorder.

In a further aspect, the present invention provides a kit comprising, in a suitable container means, a pharmaceutical composition containing a steroidal aromatase inhibitor, as an active agent, and a non-steroidal aromatase inhibitor, as a further active agent, in amounts to produce a synergistic or superadditive therapeutic activity against a sex steroid hormone-dependent disorder.

A further aspect of the present invention is to provide a method of treating a human being, particularly a female, suffering from a sex steroid hormone-dependent disorder comprising administering to said human being a steroidal aromatase inhibitor and a non-steroidal aromatase inhibitor, in amounts effective to produce a superadditive or synergistic therapeutic effect.

A further aspect of the present invention is to provide a method of treating a human being, particularly a female, suffering from a sex steroid hormone-dependent disorder comprising administering to said human being a steroidal aromatase inhibitor and a non-steroidal aromatase inhibitor, in amounts effective to produce a superadditive or synergistic therapeutic effect and to prevent resistance phenomena.

The compositions and combined therapy method of the invention provide therefore a two-way attack on cancer cell growth.

A still further aspect of the present invention is to provide a method for lowering the side effects (adverse reactions) caused by antitumor therapy with a steroidal aromatase inhibitor in a human being, particularly a female, suffering from a sex steroid hormone-dependent disorder, the method comprising administering to said human being a combined preparation comprising a steroidal aromatase inhibitor and a non-steroidal aromatase inhibitor, in amounts effective to produce a superadditive or synergistic antitumor effect, while controlling the growth of neoplasm formation.

A still further aspect of the present invention is to provide a method for lowering the side effects (adverse reactions) caused by antitumor therapy with a non-steroidal aromatase inhibitor in a human being, particularly a female, suffering from a sex steroid hormone-dependent disorder, the method comprising administering to said human being a combined preparation comprising a non-steroidal aromatase inhibitor and a steroidal aromatase inhibitor, in amounts effective to produce a superadditive or synergistic antitumor effect, while controlling the growth of neoplasm formation.

The combination preparation according to the invention can also include combination packs or compositions in which the constituents are placed side by side and can be administered simultaneously, separately or sequentially to one and the same human being. Accordingly, the steroidal aromatase inhibitor and the non-steroidal aromatase inhibitor may be present within a single or distinct container.

By the term “a superadditive or synergistic antitumor effect” as used herein is meant the inhibition of the growth tumor, preferably the complete regression of the tumor, by administering a combination of a steroidal aromatase inhibitor, as defined above, and a non-steroidal aromatase inhibitor, to a human being, particularly a human female.

Said preparation having therefore a potentiated antitumor (superadditive) activity and lower resistance occurrence with respect to products containing either a steroidal aromatase inhibitor or a non-steroidal aromatase inhibitor which is greater than the sum of the actions of individual components.

By the term “administered” or “administering” as used herein is meant any acceptable manner of administering a drug to a patient which is medically acceptable including parenteral and oral administration.

By “parenteral” is meant intravenous, subcutaneous, intradermal or intramuscular administration.

Oral administration includes administering the constituents of the combined preparation in a suitable oral form such as, e.g., tablets, capsules, suspensions, solutions, emulsions, powders, syrups and the like.

Parenteral administration includes administering the constituents of the combined preparation by subcutaneous, subcutaneous, intravenous or intramuscular injections.

The actual preferred method and order of administration of the combined preparations of the invention may vary according to, inter alia, the particular pharmaceutical formulation of the steroidal aromatase inhibitor being utilized, the particular pharmaceutical formulation of the non-steroidal aromatase inhibitor being utilized, the particular cancer being treated and the particular patient being treated. Preferably the steroidal aromatase inhibitor and the non-steroidal aromatase inhibitor are administered in a sequential or substantially simultaneous manner.

The dosage ranges for the administration of the combined preparation may vary with the age, condition and extent of the disease in the patient and can be determined by one of skill in the art.

The dosage regimen must therefore be tailored to the particular of the patient's conditions, response and associate treatments in a manner which is conventional for any therapy, and may need to be adjusted in response to changes in conditions and/or in light of other clinical conditions.

In the combined method of treatment according to the subject invention, the steroidal aromatase inhibitor may be administered simultaneously with the non-steroidal aromatase inhibitor or the compounds may be administered sequentially, in either order.

When steroidal and non-steroidal, aromatase inhibitors (or vice-versa) are given sequentially to each other, preferably the treatment starts with administration or a first aromatase inhibitor (e.g. the steroidal one) which may last from about 1 to about 3 weeks, then followed by administration of a second aromatase inhibitors (e.g. the non-steroidal one) which may last from about 1 to about 3 weeks. The above treatment schedule can be repeated up to the disease is stabilized.

An effective amount of an aromatase inhibitor antitumor agent may vary from about 0.5 to about 500 mg pro dose 1-2times a day. Exemestane, for example, may be administered orally in a dosage range varying from about 5 to about 200 mg, and particularly, from about 10 to about 25 mg, or parenterally from about 50 to about 500 mg, in particular from about 100 to about 250 mg.

Fadrozole, for example, may be administered orally in a dosage range varying from about 0.5 to about 10 mg, and particularly, from about 1 to about 2 mg.

Letrozole, for example, may be administered orally in a dosage range varying from about 0.5 to about 10 mg, and particularly, from about 1 to about 2.5 mg.

Formestane, for example, may be administered parenterally in a dosage range varying from about 250 to about 500 mg, and particularly, from about 250 to about 300 mg.

Anastrozole, for example, may be administered orally in a dosage range varying from about 0.5 to about 10 mg, and particularly, from about 1 to about 2 mg.

The therapy method according to the present invention is, in particular, suitable for treating a human being suffering from sex steroid dependent and in particular estrogen dependent disorders. Typical examples of such disorders are tumors, like ovarian, cervical and endometrial cancers in a human female or breast cancer in a human being, in particular a female.

More in particular, the combined use of a steroidal aromatase inhibitor, according to the invention, preferably exemestane, and a non-steroidal aromatase inhibitor, preferably anastrozole or letrozole, can be suitable for the treatment of patients with estrogen dependent cancers, for example, for patient with breast cancer, in particular with metastatic breast cancer.

According to a preferred aspect of the present invention the superadditive antitumor effect results in an anti breast cancer therapy having increased effectiveness in controlling, i.e. slowing, interrupting, arresting, stopping or reversing, the neoplasm formation.

As used herein, “controlling the growth” of the neoplasm refers to slowing, interrupting, arresting or stopping its growth and it does not necessarily indicate a total elimination of the neoplasm.

Therefore the term “treating” simply means that the life expectancy of an individual affected with a cancer will be increased, that one or more of the symptoms of the disease will be reduced and/or that quality of life will be enhanced.

The compositions and combined therapy method of the invention provide therefore a two-way attack on cancer cell growth.

In the compositions and method of treatment according to the invention exemestane is the most preferred example of steroidal aromatase inhibitor.

From the pharmacological point of view, the valuable biological properties of exemestane may be found in its peculiar mechanism of aromatase inactivation.

The aromatase enzyme (450ₐᵣₒₘ) is a specific form of cytochrome P450 hemoprotein composed of a P450 (heme) moiety and a peptidic moiety. The enzyme catalyzes a multistep reaction leading to aromatization of the A ring of the androgen substrate (mainly androstenedione) to estrone, requiring the presence of the cofactor NADPH.

After this enzymatic reaction, the enzyme molecule is once more available to perform a new aromatization.

The exemestane's mechanism of aromatase inhibition has been extensively studied and the compound has been found to cause enzyme inactivation. In fact exemestane, structurally related to the natural substrate androstenedione, is initially recognized by the aromatase enzyme as a false substrate, therefore competes with androstenedione at the active site of the enzyme. The compound is then transformed (through and NADPH-dependent mechanism) to an intermediate which binds irreversibly to the enzyme causing its inactivation (also known as suicide inhibition). Therefore the enzyme is definitely inactivated and de novo enzyme synthesis is required for oestrogen production.

Suitable modifications and adaptations of a variety of conditions and parameters normally encountered in clinical therapy which are obvious to those skilled in the art are within the scope of this invention.

A pharmaceutically composition containing a steroidal aromatase inhibitor and/or a non-steroidal aromatase inhibitor can be prepared according to well known techniques to those skilled in the art. For instance a pharmaceutical composition containing exemestane can be prepared according to U.S. Pat. No. 4,808,616.

1. Use of a steroidal aromatase inhibitor in the manufacture of a pharmaceutical composition for treatment of a sex steroid hormone-dependent disorder, the treatment additionally comprising the administration of a composition comprising a non-steroidal aromatase inhibitor, in amounts effective to produce a superadditive effect.

2. Use, according to claim 1, wherein the disorder is breast, cervical, ovarian and endometrial cancers.

3. Use, according to claim 2, wherein the disorder is breast cancer.

4. Use, according to claim 1, wherein the steroidal aromatase inhibitor is selected from exemestane, formestane and atamestane.

5. Use, according to claim 4, wherein the steroidal aromatase inhibitor is exemestane.

6. Use, according to claim 1, wherein the non-steroidal aromatase inhibitor is selected from fadrozole, letrozole and anastrozole.

7. Use, according to claim 6, wherein the non-steroidal aromatase inhibitor is anastrozole or letrozole.

8. Product comprising a steroidal aromatase inhibitor and a non-steroidal aromatase inhibitor, as combined preparation for simultaneous, separate or sequential administration, in amounts to produce a synergistic or superadditive therapeutic activity against an hormone-dependent disorder.

9. A product according to claim 8, wherein the combined preparation is for sequential administration, the treatment with each of the aromatase inhibitors lasting from about 1 to about 3 weeks.

10. A method of treating a human being, particularly a female, suffering from a sex steroid hormone-dependent disorder comprising administering to said human being a steroidal aromatase inhibitor and a non-steroidal aromatase inhibitor, in amounts effective to produce a superadditive or synergistic therapeutic effect.

11. A method of treating a human being, particularly a female, suffering from a sex steroid hormone-dependent disorder comprising administering to said human being a steroidal aromatase inhibitor and a non-steroidal aromatase inhibitor, in amounts effective to produce a superadditive or synergistic therapeutic effect and to prevent resistance phenomena.

12. A method for lowering the side effects (adverse reactions) caused by antitumor therapy with a steroidal aromatase inhibitor in a human being, particularly a female, suffering from a sex steroid hormone-dependent disorder, the method comprising administering to said human being a combined preparation comprising a steroidal aromatase inhibitor and a non-steroidal aromatase inhibitor, in amounts effective to produce a superadditive or synergistic antitumor effect, while controlling the growth of neoplasm formation.

13. A method for lowering the side effects (adverse reactions) caused by antitumor therapy with a non-steroidal aromatase inhibitor in a human being, particularly a female, suffering from a sex steroid hormone-dependent disorder, the method comprising administering to said human being a combined preparation comprising a non-steroidal aromatase inhibitor and a steroidal aromatase inhibitor, in amounts effective to produce a superadditive or synergistic antitumor effect, while controlling the growth of neoplasm formation.

14. A method according to claims 10 to 13, wherein the steroidal aromatase inhibitor and the non-steroidal aromatase inhibitor are administered in a sequential manner.

15. A method according to claim 14 wherein the treatment with each of the aromatase inhibitors lasts from about 1 to about 3 weeks.

16. A method according to anyone of claims 10 to 13, wherein the steroidal aromatase inhibitor and the non-steroidal aromatase inhibitor are administered in a substantially simultaneous manner.
